(19) United States
(12) Patent Application Publication
(10) Pub. No.: US 2006/0258746 A1
(43) Pub. Date: Nov. 16, 2006

(54) Oral medicament for improvement in going to sleep or waking

(75) Inventors: Toshikazu Kamiya, Tsukuba-shi (JP); Takeshi Shibasaki, Tsukuba-shi (JP); Asuka Otofuji, Tsukuba-shi (JP); Yasushi Sakai, Tsukuba-shi (JP); Akemi Ogawa, Tokyo (JP); Miho Komatsu, Tsukuba-shi (JP)

Correspondence Address: DARBY & DARBY P.C., P. O. BOX 5257, NEW YORK, NY, 10150-5257, US

(73) Assignee: Kyowa Hakko Kogyo Co., Ltd., Tokyo (JP)

(21) Appl. No.: 11/272,107

(22) Filed: Nov. 8, 2005

(30) Foreign Application Priority Data
May 13, 2005 (JP) 2005-140571

(51) Int. Cl. A61K 31/198

(52) U.S. Cl. 514/563

(57) ABSTRACT

A person, who has symptoms including difficulty in going to sleep, feeling languid after waked up or having a hard time awaking, is desirous of a medicament and a nutritional food that can alleviate the symptoms and improve his or her health. Specifically, one object of the present invention is to provide an orally administered agent, a food and drink, or a food additive which can be used for improving the process of going to sleep and waking. An orally administered agent, a food and drink, or a food additive for improving the process of going to sleep and waking, containing ornithine or a salt thereof as an active ingredient can be provided according to the present invention.

### BACKGROUND OF THE INVENTION

### Incorporation by Reference

The present application claims priority under 35 U.S.C. §119 to Japanese Patent Application No. 140571/2005 filed on May 13, 2005. The content of the application is incorporated herein by reference in its entirety.

1. Field of the Invention

The present invention is related to an orally administered agent; a food and drink; and a food additive including an active ingredient which is ornithine or a salt thereof for improving the process of going to sleep and waking.

2. Description of the Related Art

L-ornithine is being used, mostly in the U.S., as a food additive to strengthen muscle formation by letting the body secrete growth hormone or to prevent obesity by enhancing basal metabolism. Further, L-ornithine is used to produce L-ornithine-L-aspartate which is a medicament used to improve a liver disorder in Europe.

It has been reported that kale, propolis and their extracts have an affinity to the melatonin receptor and an effect to improve such as awaking disorder related to the melatonin receptor. (Japanese Published Unexamined Patent Application No. 335691/2003)

### SUMMARY OF THE INVENTION

A person, who has symptoms including difficulty in going to sleep, feeling languid after waked up or having a hard time awaking, is desirous of a medicament and a nutritional food that can alleviate the symptoms and improve his or her health. Specifically, one object of the present invention is to provide an orally administered agent, a food and drink, or a food additive which can be used for improving the process of going to sleep and waking.

The present invention relates to the following aspects (1) to (3):
(1) an orally administered agent for improving the process of going to sleep and waking which comprises ornithine or a salt thereof as an active ingredient.
(2) a food and drink, or a food additive for improving the process of going to sleep and waking, which comprises ornithine or a salt thereof as an active ingredient.
(3) a method for improving the process of going to sleep and waking which comprises administering ornithine or a salt thereof to a subject in need thereof.

### BRIEF DESCRIPTION OF THE DRAWINGS

FIG. 1 is scale graphs expressing questionnaires for evaluation using Visual Analogue Scale (VAS) method. Each end of the segment has a criterion of expression.

FIG. 2 is a graph showing improvements in going to sleep and waking by the intake of ornithine. The vertical axis shows an average improvement ratio (%) of each criterion.

### DETAIL DESCRIPTION OF THE INVENTION

Ornithine as applied in the present invention includes L-ornithine and D-ornithine, preferably L-ornithine.

Ornithine can be obtained by a chemical synthetic method or a fermentation method. Also, ornithine is commercially available.

A chemical synthetic method can be found in, for example, Coll. Czechoslov. Chem. Commun., 24, 1993 (1959).

A fermentation method is disclosed, for example, in Japanese Published Unexamined Patent Application Nos. 24096/78 and 119194/86.

L-ornithine and D-ornithine can be also purchased from, for example, Sigma Aldrich Company.

Salts of ornithine include acid addition salts; metal salts, ammonium salts, organic amine addition salts, amino acid addition salts and the like.

The acid addition salts include inorganic acid salts such as hydrochloride, hydrosulfate, nitrate and phosphate; and organic acid salts such as acetate, maleate, fumarate, citrate, malate, lactate, α-ketoglutarate, gluconate and caprylate.

The metal salts include alkali metal salts such as sodium salt and potassium salt; alkaline earth metal salts such as magnesium salt and calcium salt; aluminum salt, zinc salt and the like.

Ammonium salts include salts of ammonium, tetramethylammonium and the like.

Organic amine addition salts include salts of morpholine, piperidine and the like.

Amino acid addition salts include salts of glycine phenylalanine, lysine, aspartate, glutamate and the like.

Among the above salts, hydrochloride, citrate, malate, α-ketoglutarate and aspartate are preferably applied, but one of the remaining salts or two or more of the above salts can be arbitrarily used.

In addition to ornithine and its salt, a proper additive for each application can be added to an orally administered agent, a food and drink, or a food additive of the present invention.

The above additive includes amino acids such as valine, leucine, isoleucine, arginine, lysine, glutamine, alanine, serine, glycine, cistein and threonine, and the like.

A person can improve going to sleep or waking up by administration or ingestion of an orally administered agent, a food and drink, or a food additive according to the present invention.

The improvements in going to sleep and waking according to the present invention means providing normal sleep or normal awaking to a person who has subjective symptoms such as difficulty in going to sleep, languid feelings after waking up and a hard time awaking.

An orally administered agent according to the present invention comprises ornithine or a salt thereof, and may include one or more pharmaceutically acceptable carriers, as necessary, and another active ingredient for other medical treatments as necessary.

An orally administered agent according to the present invention can be produced by mixing ornithine or a salt thereof with carriers and the like as necessary, according to an arbitrary method well known in the technological field of pharmaceuticals.

When an orally administered agent according to the present invention is formulated, additions such as an excipient, a binder, a disintegrating agent, a lubricant, a dispersant, a suspending agent, an emulsifier, a diluent, a buffering agent, an antioxidant, and an anti-bacterial agent can be added.

Formulation of the orally administered agent includes tablets, powders, granules, emulsion, syrup, capsule and the like.

For instance, in the case of producing the orally administered agent in the form of tablets, powders, granules, or the like, the following can be added to make such formulation: excipients including a sugar such as lactose, white sugar, glucose, sucrose, mannitol or sorbitol; a starch such as potato, wheat or corn; an inorganic compound such as calcium carbonate, calcium sulfate, sodium bicarbonate or sodium chloride; and a plant powder such as licorice powder or gentian powder; disintegrating agents such as starch, agar, gelatin powder, cellulose crystal, sodium carmellose, calcium carmellose, calcium carbonate, sodium bicarbonate or sodium alginate; lubricants such as magnesium stearate, talc, hydrogenated plant oil, macrogol or silicon oil; binders such as polyvinyl alcohol, hydroxypropylcellulose, methylcellulose, ethylcellulose, carmellose, gelatin, or starch glue solution; surfactants such as fatty acid ester; plasticizers such as glycerin and the like.

In case of producing the orally administered agent in the form of a liquid preparation such as syrup and the like, the following can also be added to make such formulation: water; sugars such as sucrose, sorbitol or fructose; glycols such as polyethylene glycol or propylene glycol; oils such as sesame oil, olive oil or soybean oil; preservatives such as p-hydroxybenzoic ester; flavors such as strawberry flavor or peppermint flavor and the like.

Although the concentration of ornithine or a salt thereof can be properly selected in accordance with a kind of the orally administered agent and an effect expected by administration of the orally administered agent, it is normally in a range of 0.1 to 90% by weight of ornithine or a salt thereof; preferably in a range of 0.5 to 80% by weight, and most preferably in a range of 1 to 70% by weight.

Although a dosage of the orally administered agent of the present invention may vary depending on such factors as an administration form, and age and body weight of the person who is being administered to, for an adult per day, it is normally in a range of 50 mg to 30 g of ornithine and a salt thereof, preferably in a range of 100 mg to 10 g, and most preferably in a range of 200 mg to 3 g, which is administered one time or separately a few times. Although a period of administration is not limited, it is normally in a range of one day to one year, preferably in a range of one week to three months.

A food additive according to the present invention can be prepared by the same method applied for the above orally administered agent. The food additive is normally mixed or resolved with another food additive as necessary and processed into, for example, powders, granules, a pellet, a tablet or various solutions.

As the food or drink according to the present invention, mention may be made of the food or drink comprising ornithine or a salt thereof, or a food additive according to the present invention.

The food or drink according to the present invention can be processed and manufactured by a general food and drink manufacturing method except that ornithine or a salt thereof, or the food additive according to the present invention is added to the known food or drink.

The food or drink according to the present invention can be manufactured by a granulation method such as a fluidized-bed granulation, a stirring granulation, an extrusive granulation, a rolling granulation, an airflow granulation, a compression molding granulation, a grinding granulation, a spray granulation or a jet granulation; a coating method such as a pan coating, a fluidized-bed coating and a dry coating; an explosive puff method such as a puff dry method, an excess water vapor method, a format method or a microwave heating method; or an extrusion method using, for example, an extrusion granulation or an extruder method.

The food or drink according to the present invention includes juices; soft drinks; teas, dairy products such as lactic acid bacteria beverages, fermented milk, frozen dessert, butter, cheese, yogurt, processed milk or defatted milk; animal meat products such as ham, sausage or hamburger; fish cake products such as plate-like fish cake or kamaboko in Japanese, pipe-like fish cake or chikuwa in Japanese, or fried fish cake or satsumaage in Japanese; egg products such as rolled egg with soup or dashimaki in Japanese or egg-tofu; confectioneries such as cookie, jelly, chewing gum, candy or munch; breads; noodles; pickles; smoked food products; dried fishes; fishes boiled in soy sauce or tsukudani in Japanese; salt curing products; soups; condiments; or any other forms.

Further the food or drink of the present invention may take the forms of powdery foods; sheet-like foods; bottled foods; canned foods; retort foods; capsule foods; tablet-like foods; fluid foods; nutritious supplement drinks or the like.

The food or drink according to the present invention can be used as health foods; functional foods; nutritious supplement foods; or food for specified health use, for improving the process of going to sleep and waking up.

A food additive such as a sweetener, a coloring agent, a preservative, a thickening stabilizing agent, an antioxidant, a color fixing agent, a bleaching agent, an anti-fungous agent, a gum base, a bitter substance, an enzyme, a brightening agent, an acidifying agent, a condiment, an emulsifying agent, a reinforcing agent, a production agent, a flavor or a spice extract, which are generally used in a food and drink, can be added to the food or drink or the food additive according to the present invention.

Although the concentration of ornithine or a salt thereof can be properly selected in accordance with a kind of a food or drink, and an effect expected by administration of the food or drink, it is normally in a range of 0.1 to 90% by weight of ornithine or a salt thereof; preferably in a range of 0.5 to 80% by weight, and most preferably in a range of 1 to 70% by weight.

Although a dosage of the food or drink according to the present invention may vary depending on an administrative form; and age and body weight of the person being administered to, for an adult per day, it is normally in a range of 50 mg to 30 g of ornithine or a salt thereof, preferably in a range of 100 mg to 10 g, and most preferably in a range of 200 mg to 3 g, which is administered one time or separately a few times. Although a period of administration is not specified, it is normally in a range of one day to one year, preferably in a range of one week to three months.

The followings are test examples of the effect of ornithine on improving the process of going to sleep and waking.

### TEST EXAMPLE 1:

Ten subjects out of 28 subjects who took tablets, over 8 tablets, (Commercial Name: Remake-Ornithine, Kyowa Hakko Kogyo Co., Ltd.) in which each tablet contains 80 mg of ornithine showed improvements in waking in comparison with before administration of ornithine.

Further, 6 subjects out of 28 subjects took ornithine just before going to sleep and 3 subjects out of the 6 subjects showed an improvement in waking.

According to the test result, intaking ornithine improved the waking process, and also the intake thereof just before going to sleep was effective.

### TEST EXAMPLE 2:

Six tablets of example 1 (the tablet containing ornithine) or 6 tablets of comparative example 1 (the tablet not containing ornithine) were administered to two groups with 7 subjects out of 14 normal subjects of each male and female between 45 to 64 year a day for 3 weeks.

Before the administration and just after the administration, the improvements in going to sleep and waking of each subject were evaluated using Visual Analogue Scale (VAS) method.

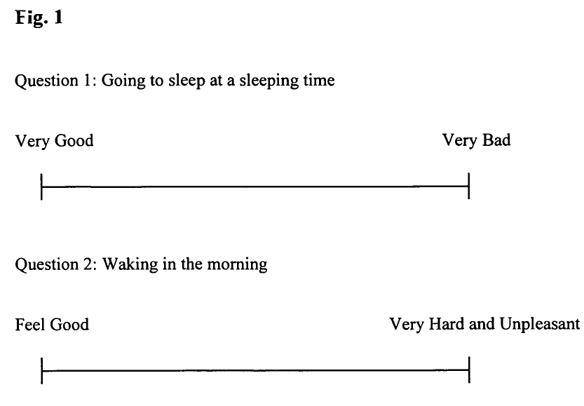

Specifically each end of the segment has a criterion of expression. Referring to FIG. 1, each subject marked somewhere in the line, corresponding to each term of the questionnaires. The distance (mm) from the left end of the line to the marked point was measured and the difference between before and after the test was calculated. The difference by the value before the test was shown in the percentage calculated; and each average value and standard deviation for each group were calculated. Further the average improvement ratio (%) was the value obtained by subtracting the average value of the placebo group from the average value of ornithine group. Further, it was confirmed that there was no difference between two groups before the administration test.

Further the test was carried out under a random assignment and the comparison between the double blind parallel groups was carried out. The test of the statistically significant difference between two groups was an unpaired t-test of both side distributions using the difference between the beginning and just after the test.

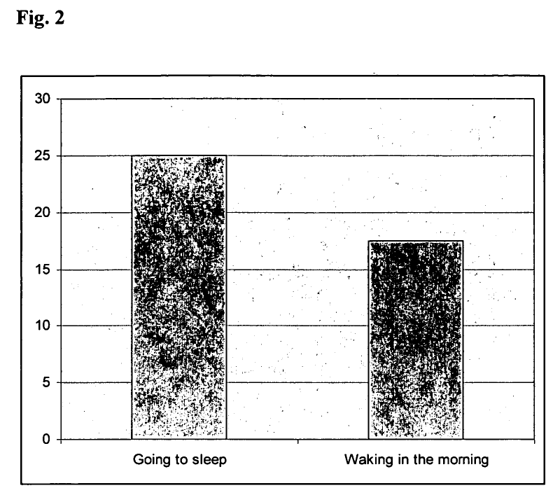

The results are shown in FIG. 2. Every term showed that there was the intake effect of ornithine. Specifically, the results showed that not only an improvement in the waking process was evident but also an improvement in going to sleep were obtained by the intake of ornithine.

The followings are the example of the present invention.

### EXAMPLE 1

### Production of a Tablet Containing Ornithine

A mixture of 136.2 Kg of ornithine hydrochloride (Commercial name: L-ornithine hydrochloride, Kyowa Hakko Kogyo Co., Ltd.); 136.0 Kg of a fine cellulose crystal (Commercial name: Avicel FD101, Asahi Kasei Chemicals Co., Ltd.); 6.6 Kg of sucrose fatty acid ester (Commercial name: DK ester F-20W, Daiichi Kogyo Seiyaku Co. Ltd.); 1.2 Kg of calcium phosphate (Commercial name: Tricalcium phosphate, Taihei Chemical Industrial Co., Ltd.); and 20.0 Kg of β-cyclodextrin (Commercial name: Seldex B-100, Nihon Shokuhin Kako Co., Ltd.); was mixed using a conical blender (CB-1220 Blender, Nihon Kansoki Co., Ltd.). The mixture obtained was compressed and molded to a tablet of 250 mg with 8 mm of diameter under 10 KN of compression-molding pressure using the rotary compression molding machine (VIRG0524SS1AY, Kikusui Seisakusyo Ltd.).

### EXAMPLE 2

### Production of an Enteric Capsule Containing Ornithine

A mixture of 20 Kg of the mixture produced in Example 1 and 0.2 Kg of silicon dioxide was mixed and stirred. The mixture obtained was put into a capsule-filling machine to fill 20,000 tablets of gelatin Number 2 hard-capsules to provide the hard-capsules. The surfaces of the hard-capsules obtained were coated with a zein solution using High Coater HCT-48 (Freund Corporation) to produce 20,000 enteric capsules containing ornithine hydrochloride.

### EXAMPLE 3

### Production of Enteric Tablet Containing Ornithine

The surfaces of the tablets produced in Example 1 were coated with shellac solution using High Coater HCT-48 (Freund Corporation) to produce an enteric tablet.

### EXAMPLE 4

### Production of a Drink Containing Ornithine

Each 1.28 Kg of ornithine hydrochloride (Commercial name: L-ornithine hydrochloride, Kyowa Hakko Kogyo Co. Ltd.); 3 Kg of erythritol (Nikken Kagaku Co. Ltd.); 0.05 Kg of citric acid (Kyowa Hi Foods Co. Ltd.); 3 g of artificial sweetener; and 0.06 g of flavor were stirred and dissolved in 50 L of water at solution temperature 70° C.; After the pH of the solution was adjusted to 3.3, the solution was sterilized using plate sterilization and filled into bottles. The bottle was sterilized using a pasteurizer to produce the ornithine beverage.

### COMPARATIVE EXAMPLE 1

Instead of ornithine hydrochloride in Example 1, the same amount of lactose was used to produce a tablet not containing ornithine.

While the invention has been described in detail and with reference to specific embodiments thereof, it will be apparent to one skill in the art that various changes and modifications can be made therein without departing from the spirit and scope thereof. All references cited herein are incorporation in their entirety.

What is claimed is:

1. An orally administered agent for improving the process of going to sleep and waking, which comprises ornithine or a salt thereof as an active ingredient.

2. A food and drink or a food additive for imporiving the process of going to sleep and waking, which comprises ornithine or a salt thereof as an active ingredient.

3. A method for improving the process of going to sleep and waking, which comprises administrating ornithine or a salt thereof to a subject in need thereof.
